(19) United States
(12) Patent Application Publication
(10) Pub. No.: US 2025/0114097 A1
(43) Pub. Date: Apr. 10, 2025

(54) REDUCER FOR CRITICAL LIMB ISCHEMIA AND CRITICAL HAND ISCHEMIA

(71) Applicant: Cardiovascular Systems, Inc., St. Paul, MN (US)

(72) Inventors: Brad J. Martinsen, St. Louis Park, MN (US); Zsuzsanna Igyarto, Philadelphia, PA (US); Erik D. Eli, Redwood City, CA (US)

(21) Appl. No.: 18/906,647

(22) Filed: Oct. 4, 2024

Related U.S. Application Data
(60) Provisional application No. 63/677,190, filed on Jul. 30, 2024.
(60) Provisional application No. 63/542,610, filed on Oct. 5, 2023.

(51) Int. Cl. A61B 17/12; A61B 17/00

(52) U.S. Cl. CPC A61B 17/12036; A61B 17/1204; A61B 17/12109; A61B 17/12172; A61B 2017/00004; A61B 2017/00778; A61B 2017/1205

(57) ABSTRACT

A method of treatment of critical limb ischemia within a foot of a patient and/or critical hand ischemia within a hand of a patient, the method including the steps of delivering a venous reducer by way of the patient's vasculature to a first delivery location within a vein of the foot and/or hand, the venous reducer comprising an expandable structure that when expanded includes an upstream portion and a downstream portion that are connected by a narrowed connection portion, and expanding the venous reducer within the vasculature of the foot and/or hand of the patient and thus creating an hourglass shaped structure within the vasculature where the narrowed connection portion acts as a restriction for blood flow

### CROSS-REFERENCE TO RELATED APPLICATIONS

This application claims the benefit of U.S. Provisional Patent Application No. 63/542,610, filed Oct. 5, 2023 and U.S. Provisional Patent Application No. 63/677,190, filed Jul. 30, 2024, the entire contents of which are incorporated herein by reference in their entireties.

### TECHNICAL FIELD

The present invention is directed to medical device implants to be inserted within the venous system of the body and treatments for chronic or critical limb ischemia and critical hand ischemia.

### BACKGROUND

Critical Limb Ischemia “CLI” is symptomatic as foot pain when the foot is at rest (foot angina), the non-healing of wounds on the foot, or gangrene (tissue necrosis). Foot pain can be a burning pain in the ball of the foot and the toes that is typically worse at night due to the loss of gravity assisted blood flow to the foot, as such is farthest from the heart. Occlusions within the arteries within the leg or foot can cause a reduction in blood flow to the foot, which in turn can result in CLI symptoms.

The foot includes arterial vasculature and microvasculature that is responsible for delivery of oxygenated blood to tissues of the foot (arterial blood flow) and for returning deoxygenated blood back to the heart (venous blood flow). With arterial vascular occlusion, oxygenated blood flow is reduced and thus the foot tissues do not get adequate oxygen delivery and cellular waste removal.

Similarly, the hand includes arterial vasculature and microvasculature that is responsible for delivery of oxygenated blood to tissues of the hand (arterial blood flow) and for returning deoxygenated blood back to the heart (venous blood flow). With arterial vascular occlusion, oxygenated blood flow is reduced and thus the hand tissues do not get adequate oxygen delivery and cellular waste removal.

Venous return of deoxygenated blood from the foot and/or hand to the heart is assisted in several ways, including a respiratory pump action and skeletal muscle pumps, and with regard to the foot, ground reaction forces on the foot during standing and walking for creating what is known as a foot or plantar pump. Functional vein valves (one-way valves) are also an important part of good venous blood flow by preventing blood back flow during a resting cycle.

A plantar pump works as the plantar surface of the foot deforms during walking allowing the heel and forefoot to be used as compression pumps that expel blood into the calf veins and toward the heart. Specifically, ground forces during walking act on the plantar veins to pump blood into the calf veins, such as the tibial and fibular veins, and the vein valves prevent back flow into the foot.

A medical device implant has been developed for implantation within the coronary sinus of the heart, the purpose of which is to restrict blood flow into the right atrium and increase blood pressure within the coronary sinus. The device comprises a coronary sinus reducer that can be a balloon-expandable device (using materials such as stainless steel, for example) or a self-expanding device (using materials such as nitinol, for example). Once deployed and expanded, the device has an hourglass shape. Blood flow is disrupted as flow is limited by the narrow central orifice of the reducer, which acts as a restriction. Blood pressure on the upstream side (closer to the capillaries) of the restricted waist of the reducer is increased relative to the blood pressure on the downstream side of the reducer. One purpose is to counter spasmic pressure of diseased coronary vessels for relieving angina pectoris.

### SUMMARY

In an embodiment, the present invention is directed to a medical implant that comprises a venous reducer for the foot and methods of implantation of such a venous reducer within the foot. An object of such a venous reducer for the foot is to control blood flow in situations where there is poor microvasculature blood flow within the foot or a part of the foot, wounds on the foot that won't heal, imminent limb loss, or foot angina. Such a venous reducer can cause back pressure and reverse flow within small blood vessels within the foot and/or increase the size of the microvasculature to re-establish blood flow in areas of reduced flow within the foot for relieving pain, inducing angiogenesis of new blood vessels, countering spasmic pressure of small blood vessel disease, and keeping blood and thus oxygen in the foot or a part of the foot for longer to promote wound healing.

More specifically, it is an object of the invention to implant a venous reducer within the foot or calf to regulate the venous plantar pump for enabling physicians to improve the treatment of chronic or critical limb ischemia in conjunction with peripheral vascular intervention (PVI) of target arterial vessels in the leg or foot. PVI can be done by various techniques including peripheral angioplasty, drug-coated balloons, stents, scaffolds, arterial thrombectomy, or peripheral atherectomy. The plantar pump is comprised of the posterior tibial vein (PTV), lateral plantar vein (LPV), and the medial plantar vein (MPV). This implantable venous reducer device will create a restrictive and/or venturi effect in the foot when implanted at the bifurcation of the plantar pump. A higher pressure will form in the foot venous system versus the above-the-ankle venous system, causing venous blood to push back across the capillaries and microvasculature of the forefoot and digits. The higher pressure in the proximal portion of the venous plantar pump (LPV and MPV) will: (1) keep capillary blood in the foot longer, allowing any remaining oxygen to be utilized, (2) drive pressure into the microvasculature, opening collapsed arteries (dormant/hibernating), allowing better arterial blood flow into the foot, and (3) the resulting increased amount of oxygen in the foot will decrease foot angina, promote angiogenesis, and improve wound healing. It is further contemplated to create a tunable venous reducer via Wi-Fi or other communication signal so normal vein flow could be restored at will by the physician.

In another embodiment, the present invention is directed to a medical implant that comprises a venous reducer for the hand and methods of implantation of such a venous reducer within the venous system of the hand. An object of such a venous reducer for the hand is to control blood flow in situations where there is poor microvasculature blood flow within the hand or a part of the hand, wounds on the hand that won't heal, imminent limb loss, or hand angina. Such a venous reducer can cause back pressure and reverse flow within small blood vessels within the hand and/or increase the size of the microvasculature to re-establish blood flow in areas of reduced flow within the hand for relieving pain, inducing angiogenesis of new blood vessels, countering spasmic pressure of small blood vessel disease, and keeping blood and thus oxygen in the hand or a part of the hand for longer to promote wound healing.

### BRIEF DESCRIPTION OF THE DRAWINGS

The present invention will be further explained with reference to the appended Figures, wherein like structure is referred to by like numerals throughout the several views, and wherein:

FIG. 1 is a top view of a venous reducer;

FIG. 2 is a schematic top view of the venous reducer of FIG. 1 positioned relative to a delivery catheter having a single balloon;

FIG. 3 is a schematic top view of the venous reducer of FIG. 1 positioned relative to a delivery catheter having two spaced balloon portions;

FIG. 4 is a schematic of the venous vasculature of a foot as viewed from the top of the foot;

FIG. 5 is a schematic of the venous vasculature of a foot as viewed from the bottom of the foot;

FIG. 6 is a schematic of a vein as has been shaped by implantation of a venous reducer of the type illustrated in FIG. 1;

FIG. 7 is a schematic of the venous vasculature of a hand as viewed from the top of the hand;

FIG. 8 is a simplified schematic ventral (anterior) view of the venous vasculature of the hand and forearm;

FIG. 9 is a front view of a woven cylindrical element for use in making a venous reducer;

FIG. 10 is a front view of an intermediate step in the forming of a venous reducer with wire/heat resistant thread;

FIG. 11 is another front view of an intermediate step in the forming of a venous reducer;

FIG. 12 is an hourglass-shaped tool of the type used in the forming of a venous reducer;

FIG. 13 is a perspective view of a venous reducer after having the wire/heat resistant thread removed;

FIGS. 14a-14c are schematic views of exemplary steps for deploying a venous reducer; and

FIGS. 15a-15d are schematic views of exemplary steps for deploying a venous reducer.

### DETAILED DESCRIPTION

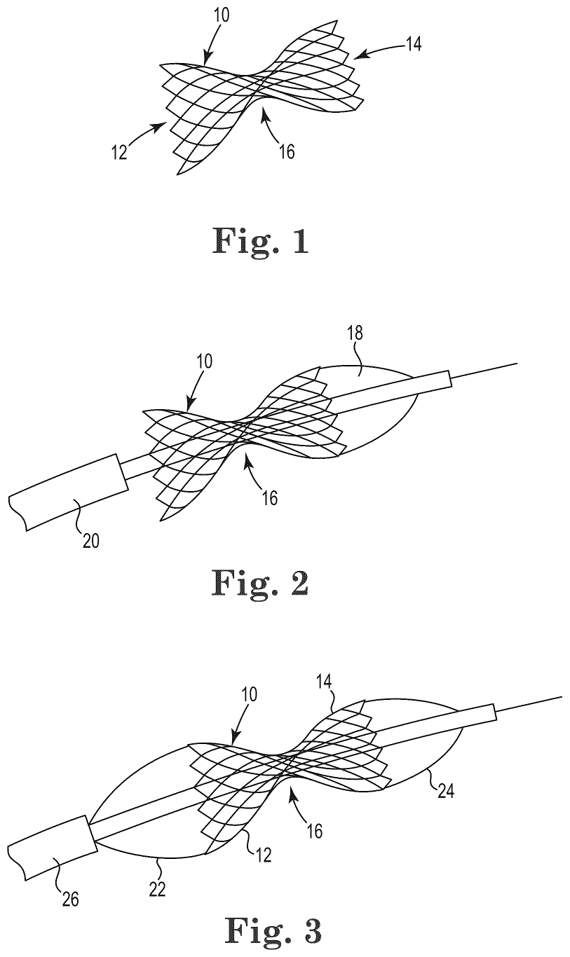

With reference to the drawings, wherein like features are labeled with like characters throughout the several figures, and initially to FIG. 1, a venous reducer 10 in accordance with the present invention is illustrated. The venous reducer 10 is an implantable device to be implanted within a venous system of the foot, as discussed in greater detail below. The venous reducer 10 can comprise a structure similar to known stents, such as can be comprised of stainless-steel or cobalt alloys (with or without a covering of fabric or graft material), nitinol (laser cut or wire interwoven), or the like. The structure is preferably expandable to be inserted to a specified location within the vasculature of the foot or ankle, such as delivered by a catheter in a conventional way. The venous reducer 10 can be expandable by one or more balloons or may be self-expandable if made from a material such as nitinol.

The venous reducer 10 is preferably hourglass shaped, as shown in FIG. 1, with an upstream portion 12 and a downstream portion 14 divided from one another by a narrow connection portion 16. The structure of the venous reducer 10 can comprise metal wires that are interlaced with one another as known in stent structure creation or can comprise a lattice of metal elements as also known for stent structure creation. Whatever structure is used, the upstream portion 12 is to be larger in an expanded diameter than the narrow connection portion 16. Likewise, the downstream portion 14 is preferably also larger in its expanded diameter than the narrow connection portion 16 and may be similar in shape and size as the upstream portion 12, but may be slightly or substantially different in shape and/or size. A flared section adjacent to the proximal and/or distal end may assist in anchoring the reducer in the venous system to minimize risk of embolization. The narrow connection portion 16 creates a restriction between the upstream portion 12 and the downstream portion 14, which in turn increases fluid pressure such as blood pressure on the upstream side of the venous reducer 10 as compared to the downstream side. The purpose for this as a medical procedure will be described in greater detail below.

As described above, an embodiment of the venous reducer 10 can comprise a nitinol structure or woven structure that self-expands at body temperature after delivery to a desired location within the foot or ankle, such as via a catheter utilizing a guide wire inserted along the vasculature to the delivery site, as is known. The structure can comprise a tube-shaped implant that is compressed and held in a compressed state by a sleeve or other structure during insertion. Once at the delivery location, the sleeve or other limiting component (not shown) can be manipulated to permit expansion of the venous reducer in a desired position. Upon expansion, the venous reducer 10 can be maintained at the implant site (i.e., landing zone) by friction of the expanded structure of the venous reducer 10 or by placing the reducer at the location of a valve to anchor the implant to the vasculature. The narrow connection portion 16 can be formed as part of the implant structure prior to being compressed for insertion, such as by designing a wire interlacing or lattice with the narrow connection portion 16, or the narrow connection portion can be formed by limiting its expansion after delivery, such as by having a restricting wire or band at a central location of the venous reducer 10 structure. In any case, it is desirable to create the hourglass overall structure as discussed above.

The venous reducer 10 can instead be expanded at the implant site by deforming a compressed structure, such as by a balloon as is well known for stent delivery and expansion. If a single balloon 18 is provided as along a delivery catheter 20 (FIG. 2), the balloon can expand either the upstream portion 12 or the downstream portion 14 first, followed by deflation and subsequent re-positioning and expanding the other while maintaining the narrow connection portion 16 unexpanded. FIG. 2 shows the expansion of the downstream portion 14 after expansion of the upstream portion 12. If two balloons 22 and 24 are provided along such a catheter 26 (FIG. 3) the balloons 22 and 24 can be provided at spaced locations along the distal end of the catheter 26 so that they expand the upstream and downstream portions 12 and 14 at the same time while leaving the narrow connection portion 16 unexpanded. In another alternative, a single balloon having an hourglass shape can be utilized to simultaneously expand the upstream and downstream portions 12 and 14 without expanding the connection portion 16. It is also contemplated that the narrow connection portion may be designed similar to the nitinol design discussed above such that that narrow portion 16 is not able to expand as may be limited by its structural design or an additional wire, band, or other restricting component.

The venous reducer 10 could be used in conjunction with arterial peripheral vascular intervention (PVI), such PVI techniques including peripheral angioplasty, drug-coated balloons, stents, scaffolds, arterial thrombectomy, or peripheral atherectomy, or the like as known or as developed. Additionally, if a balloon is used to deploy the reducer 10, a therapeutic drug agent (e.g., human growth factor agents or anti-thrombogenic) may be used. The PVI technique can be used on the inflow and/or outflow arteries of the leg to improve arterial blood flow into the foot. The reducer 10 is placed in the venous area of the foot to cause back pressure in the microvasculature of the foot to allow that improved arterial blood flow via PVI to move through the foot.

Veins have less elastic material than arterial vessels and thus do not keep their shape without blood flow. Thus, regardless of the reducer material, the vein wall will eventually collapse over the reducer and endothelial cells of the vein wall will cover the reducer material. The timing of a full reducer effect post-placement, however, will depend on the reducer design. For example, covered reducers (as mentioned above in similar stent making and usage, and which may include porous expandable coverings such as electrospinning or ePTFE, for example) will immediately cause a pressure change, whereas un-covered reducers may require a few months for cells of the vascular system to grow out over the reducer structure, covering the openings of the reducer structure and further blocking blood flow so the vein will completely collapse on the hourglass shaped reducer or simply limit the blood flow to the central portion of the reducer. Thus, a full effect would take place once that process is complete. However, it has been reported in the coronary sinus medical experience that patients achieve immediate angina relief despite the use of an uncovered-stent reducer. This implies that veins may immediately collapse over the hourglass shaped stent structure regardless of the reducer design (covered or uncovered). In patients with severe wounds (imminent limb loss), a covered-reducer system could accelerate wound healing, while a non-covered reducer could be better suited for patients with only foot angina to alleviate pain and prevent wounds from developing.

It is also contemplated that the venous reducer 10 can comprise a bioabsorbable material that is absorbed into the vasculature at a desired period after implantation. For example, a bioabsorbable semi-crystalline polymer material, such as the poly-L-lactic acid (PLLA), is known to be used for cardiac (i.e., Absorb) and below-the-knee stents (e.g., Esprit BTK) from Abbott Vascular. Other suitable bioabsorbable materials for the reducer 10 include polyglycolic acid (PGA) and blends of PGA and PLLA, along with metal stents made of an absorbable magnesium. The use of such a bioabsorbable material for the venous reducer would allow a physician to temporarily treat a CLI of the foot for a desired period to allow healing, after which unrestricted blood flow can ensue and can eliminate the possible need to remove an implant in the foot region.

It is also contemplated that such a venous reducer 10 can be designed to be removable after a desired treatment period, such as six months after implantation. The following compounds could be used to make the venous reducer 10 retrievable: heparin, diamond-like carbon coating, hydrogel, PTFE, antibody coating (prevent adherence of cells), biomimetic nanostructured coating (prevent cell adhesion), phosphorus-32 (ionizing radiation, or titanium nitride-oxide coating. Most CLI wounds heal between 3-6 months so the reducer could be removed within this 3-6-month period, but it is understood a longer or shorter period can also be considered depending on the seriousness of the wound.

Moreover, percutaneous stent retrieval can be successfully achieved using several techniques including a small-balloon technique, a double-wire technique, or a loop snare.

In cases where the initial placement of an expanded stent does not provide the desired results to the patient, a secondary balloon may be expanded within the stent to further open the reducer, thereby establishing or re-establishing a desired blood flow.

As above, the material for the reducer 10 may comprise a superelastic (i.e., shape memory) material such as Nitinol that self-expands and can be delivered without the need for a balloon. Also optionally, the reducer may be comprised of a non-shape memory material, such as stainless steel that is woven so that the structure of the implant is self-expanding due to the weave and the material properties of the stainless steel or cobalt chrome (e.g. it does not plastically deform or take an undue set when compressed into a delivery catheter).

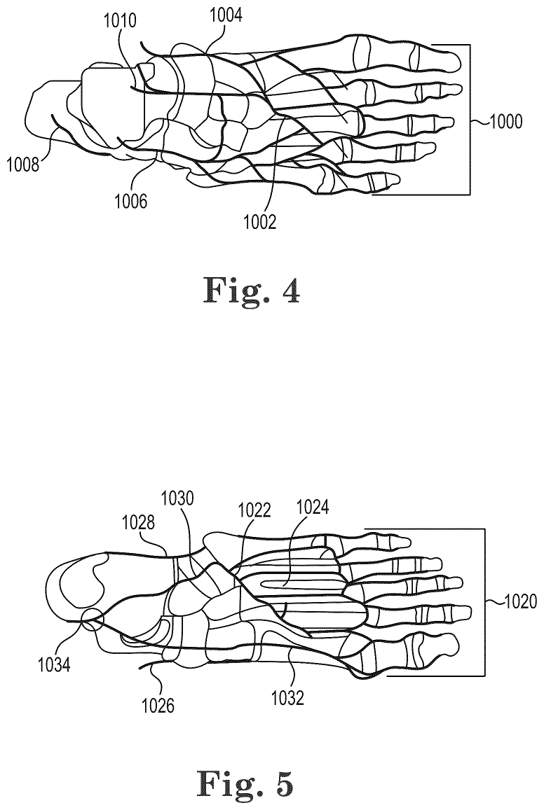

FIG. 4 is a schematic of the venous vasculature of the foot looking at the top of the foot. The most distal veins in the foot are the dorsal digital veins (DDV) 1000 that extend along the digits of the dorsal side of the foot. The dorsal digital veins (DDV) 1000 connect with other veins central in the foot including the dorsal venous arch (DVA) 1002, the long saphenous vein (LSV) 1004, and the short saphenous vein (SSV) 1006. Finally, the fibular vein (FV) 1008 and the anterior tibial veins (ATV) 1010 extend across the ankle and up the leg.

FIG. 5 is a schematic of the venous vasculature of the foot looking at the bottom of the foot. Again, the most distal veins are plantar digital veins (PDV) 1020 that connect with to central foot veins including the plantar venous arch (PVA) 1022, the dorsal venous arch (DVA) 1024 (below the bone structure), the long saphenous vein (LSV) 1026, the short saphenous vein (SSV) 1028, the lateral plantar vein (LPV) 1030, and the medial plantar vein (MPV) 1032. The lateral plantar vein (LPV) 1030 and the medial plantar vein (MPV) 1032 join at the posterior tibial vein (PTV) 1034 at the ankle from which the posterior tibial vein (PTV) 1034 extends up the leg.

The plantar pump is comprised of the posterior tibial vein (PTV) 1034, the lateral plantar vein (LPV) 1030, and the medial plantar vein (MPV) 1032. This implantable venous reducer 10 will create a restrictive and/or venturi effect in the foot when implanted at the bifurcation of the plantar pump as seen within the circle 1036 of FIG. 5.

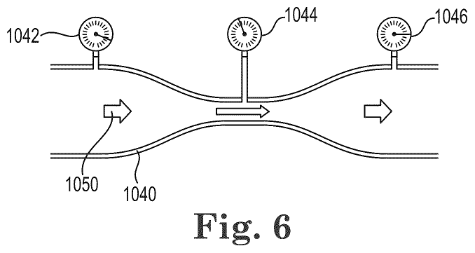

As shown schematically in FIG. 6, a vein 1040 within which a venous reducer 10 is implantable is shaped by venous reducer 10 to be similar, as discussed above. Blood flows in a direction 1050 from an area of greater pressure (e.g. in the foot of a patient) to an area that will have a lower pressure (e.g., above the ankle of a patient). Pressure gauge 1042 illustrates pressure at an upstream side of vein 1040, pressure gauge 1044 illustrates pressure at the area in which a venous reducer is placed, and pressure gauge 1046 illustrates pressure at a downstream side of vein 1040. Blood flow on the upstream side of the narrowed vein is of the greatest pressure within the region of the vein 1040 because of the restriction and/or venturi created at the narrowed vein, which higher pressure would be within the foot vasculature with the venous reducer implanted at the plantar pump. The pressure at the restriction and/or venturi would be the least pressure within this region of the vein 1040. Blood flow on the downstream side of the narrowed vein will be less than on the upstream side also because of the restriction and/or venturi created by the narrowed vein. This lessened pressure would be experienced above the ankle with the venous reducer 10 implanted at the plantar pump. Similar effects can be created elsewhere in the foot, at or near the ankle, or above the ankle in the lower leg as other examples.

Referring again to FIG. 5, a higher pressure will form in the foot venous system versus the above-the-ankle venous system, causing venous blood restriction across the capillaries of the microvasculature of the forefoot and digits. The higher pressure in the proximal portion of the venous plantar pump (LPV 1030 and MPV 1032) will: (1) keep venous blood in the foot longer, allowing any remaining oxygen to be utilized and allow oxygen to transfer across the capillaries at a higher pressure, (2) drive pressure into the microvasculature, opening collapsed arteries (dormant/hibernating), allowing better arterial blood flow into the foot, and (3) the resulting increased amount of oxygen in the foot will decrease foot angina, promote angiogenesis, and improve wound healing.

It is also contemplated that such a venous reducer may be delivered and located at other locations of the foot. For example, a venous reducer 10 can be positioned to affect only a portion of the foot. If for example, the CLI were more in the digits of the foot, such a venous reducer could be positioned closer to the digits, such as the plantar venous arch (PVA) 1022, lateral plantar vein (LPV) 1030 (distal portion closer to the digits), medial plantar vein (MPV) 1032 (distal portion closer to the digits), posterior tibial vein (PTV) 1034 (above the ankle), anterior tibial vein (ATV) 1010 (above the ankle), or any combination of these veins depending on where the pain or wound is located. Thus, multiple venous reducers 10 could be placed in one patient if needed. Also, if CLI is in one limb, it is more than likely to be present in the other limb. Thus, a venous reducer 10 could be also placed in the other limb to prevent wounds.

Another particular use for a venous reducer as described herein is for treatment of critical hand ischemia (CHI). CHI is a severe condition characterized by insufficient blood flow to the hands. Left untreated, it can lead to tissue damage, chronic pain, and even amputation. As such, another embodiment of the present invention is directed to a medical implant that comprises a venous reducer for the hand and methods of implantation of such a venous reducer within the hand that can be utilized to alleviate CHI when implanted in the venous system of the hand. An object of such a venous reducer for the hand is to control blood flow in situations where there is poor microvasculature blood flow within the hand or a part of the hand, wounds on the hand that won't heal, imminent limb loss, or hand angina. By creating a controlled narrowing or constriction within the vein, the venous reducer increases venous pressure, which in turn creates high pressure in the small vessels of the digits, opening vessels that had previously closed. The treatment may also promote the formation of collateral circulation. This improved circulation enhances blood flow to the ischemic areas of the hand, alleviates symptoms, promotes angiogenesis, counters spasmic pressure of small blood vessel disease, and keeps blood and thus oxygen in the hand or a part of the hand for longer to promote wound healing.

Referring again to FIGS. 1-3, venous reducer 10 is an implantable device to be implanted within a venous system of the hand. Although the sizing and configuration of the device can be like that described above relative to implantation of venous reducer 10 into an area of the foot, venous reducer 10 can be sized and configured at least slightly differently for implantation of venous reducer 10 into an area of the hand for treating CHI. In any case, the description of venous reducer 10 described above generally applies also to the use of such a device within the hand area, including the materials, structures, techniques, and variations thereof. The structure of the venous reducer for treatment of CHI is thus also preferably expandable to an hourglass shape and designed to be inserted to a specified location within the vasculature of the hand or wrist, such as delivered by a catheter in a conventional way. The venous reducer 10 for use in the area of the hand can be expandable by one or more balloons or may be self-expandable.

The venous reducer 10 could be used in conjunction with arterial peripheral vascular intervention PVI in the hand area, such PVI techniques including peripheral angioplasty, drug-coated balloons, arterial thrombectomy, or peripheral atherectomy, or the like as known or as developed. Additionally, if a balloon is used to deploy the reducer 10, a therapeutic drug agent may be used. The PVI technique can be used on the inflow and/or outflow arteries of the arm to improve arterial blood flow into the hand. The reducer 10 is placed in the venous area of the hand to cause back pressure in the microvasculature of the hand to allow that improved arterial blood flow via PVI to move through the hand.

As described relative to placement of a venous reducer in the foot area, it is also contemplated that use of the venous reducer 10 for treatment of the hand can comprise a bioabsorbable material that is absorbed into the vasculature at a desired time period after implantation. For example, a bioabsorbable semi-crystalline polymer material, such as the poly-L-lactic acid (PLLA), is known to be used for cardiac (i.e., Absorb) and below-the-knee stents (e.g., Esprit BTK) from Abbott Vascular. Other suitable bioabsorbable materials for the reducer 10 include polyglycolic acid (PGA) and blends of PGA and PLLA, along with metal stents made of an absorbable magnesium. The use of such a bioabsorbable material for the venous reducer would allow a physician to temporarily treat a CHI for a desired period after which unrestricted blood flow can ensue and can eliminate the need to remove an implant in the hand region.

It is also contemplated that such a venous reducer 10 for use in treating CHI can be designed to be removable after a desired treatment period, such as six months after implantation. The following compounds could be used to make the venous reducer 10 retrievable: heparin, diamond-like carbon coating, hydrogel, PTFE, antibody coating (prevent adherence of cells), biomimetic nanostructured coating (prevent cell adhesion), phosphorus-32 (ionizing radiation, or titanium nitride-oxide coating. Most CHI wounds heal between 3-6 months so the reducer could be removed within this 3-6-month period, but it is understood a longer or shorter period can also be considered depending on the seriousness of the wound.

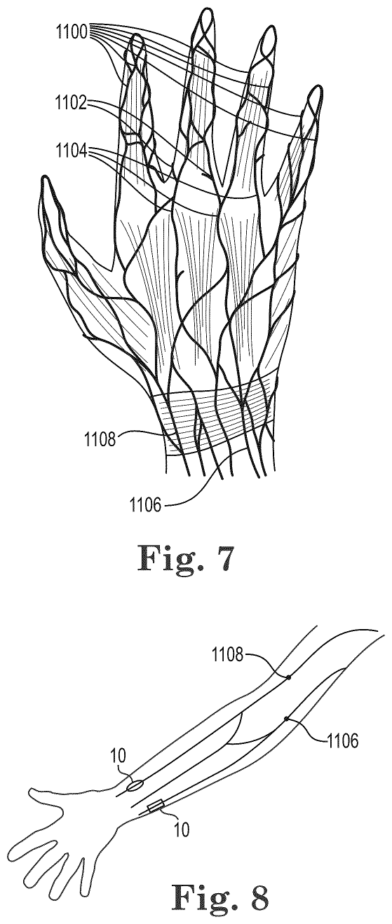

FIG. 7 is a schematic ventral (anterior) view of the venous vasculature of the hand and wrist. The most distal veins in the hand are the dorsal digital veins (DDV) 1100 that extend along the digits of the dorsal side of the hand. These veins connect to the intercapitular veins (IV) 1102 and dorsal metacarpal veins (DMV) 1104. The dorsal venous network more specifically includes a dorsal digital vein (DDV) 1100 from the radial side of the index finger and one from the ulnar side of the little finger, and both digital veins (DDV) 1100 of the thumb. This dorsal veinous network connects with the basilic vein (BV) 1106 and the cephalic vein (CV) 1108 and extend across the wrist and up the forearm.

FIG. 8 is a simplified schematic dorsal (backside) view of the venous vasculature of the hand and forearm, particularly illustrating two exemplary locations for venous reducers of the invention. For one example, a venous reducer 10 is positioned in the cephalic vein (CV) 1108 near the wrist. For another example, a venous reducer 10 is positioned in the basilic vein (BV) 1106 near the wrist. It is possible for venous reducers to be positioned in one or both of the cephalic vein and the basilic vein for a particular patient, depending on the desired results.

In some cases, the cephalic vein could be the best vein for placing venous reducer 10 to treat CHI due to its anatomical characteristics and accessibility (e.g., superficial, adequate size and diameter, well-connected with other venous structures in the arm and hand, etc.). However, in cases where the cephalic vein is not suitable or is otherwise not preferred, the venous reducer 10 can instead be placed in the basilic vein. Although the basilic vein has a larger diameter than the cephalic vein and has a high volume of blood flow, it is not as superficial such that accessing it can be more challenging.

For CHI treatment, a preferable location for the venous reducer implantation is near the wrist rather than near or above the elbow, as shown in FIG. 8, as the increased venous pressure can more directly affect the hand and fingers and more effectively promote collateral circulation in the hand. However, in other cases, the venous reducer 10 can be implanted closer to the elbow than illustrated, or even closer to the wrist. In addition, the distal part of the cephalic vein near the wrist is more superficial and accessible for the implantation.

Referring again to FIG. 6, the vein 1040 within which a venous reducer 10 is implantable is shaped by the venous reducer 10 to be similar, as discussed above. Blood flow on the upstream side of the narrowed vein is of the greatest pressure within the region of the vein 1040 as a result of the restriction and/or venturi created at the narrowed vein, which higher pressure would be within the hand vasculature with the venous reducer implanted at the cephalic vein. The pressure at the restriction and/or venturi would be the least pressure within this region of the vein 1040. Blood flow on the downstream side of the narrowed vein will be less than on the upstream side also because of the restriction and/or venturi created by the narrowed vein. This lessened pressure would be experienced above the wrist with the venous reducer 10 implanted at the cephalic vein. Similar effects can be created elsewhere in the hand, at or near the wrist, or above the wrist in the forearm as other examples.

Referring again to FIGS. 7 and 8, a higher pressure will form in the hand venous system versus the above-the-wrist venous system, causing venous blood restriction across the capillaries and microvasculature of the palm and digits. The higher pressure will: (1) keep venous blood in the hand longer, allowing any remaining oxygen to be utilized and allow oxygen to transfer across the capillaries at a higher pressure, (2) drive pressure into the microvasculature, opening collapsed arteries (dormant/hibernating), allowing better arterial blood flow into the hand, and (3) the resulting increased amount of oxygen in the hand will decrease hand angina, promote angiogenesis, and improve wound healing.

It is also contemplated that the venous reducers described herein may be delivered and located at other locations of the hand. For example, a venous reducer 10 can be positioned to affect only a portion of the hand. If for example, the CHI was more in the digits of the hand, such a venous reducer could be positioned closer to the digits, such as the dorsal metacarpal veins or the intercapitular veins or a combination of these veins, depending on where the pain or wound is located. Thus, multiple venous reducers 10 could be placed in one patient if needed. Also, if CHI is in one hand/arm, it is more than likely to be present in the other hand/arm. Thus, a venous reducer 10 could be also placed in the other hand/arm to prevent wounds.

It is further contemplated to create a tunable venous reducer 10 that can be changed in situ via Wi-Fi, Bluetooth or other communication signal so that blood flow in any portion of the venous could be reduced by the venous reducer or restored to unreduced blood flow at will by the treating physician. Such a system would require at least an additional component that can be activated, for example to create the narrow connection portion 16 at will. If using a self-expanding structure for the venous reducer 10, a wire or band or the like could be triggered by a signal to cause the narrowing of the connection portion 16 that may be released so that the connection portion 16 expands similarly to the upstream and downstream portions 12 and 14. A subsequent signal could likewise create the narrowing by tightening such a wire, band or the like.

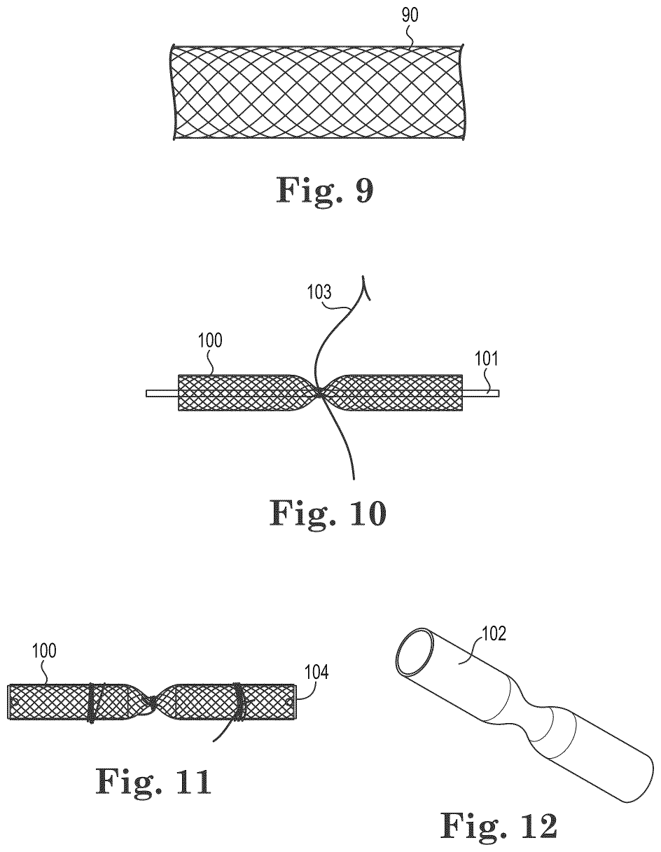

Referring now to FIGS. 9-13, one exemplary method is shown of manufacturing a venous reducer of the type described herein for placement in the venous system of a foot, hand, and/or other areas of the body. Although similar methods can be used with different materials for different types of venous reducers (e.g., balloon-expandable venous reducers), a braided Nitinol cylinder 90 is shown in FIG. 9, which is shapable into a self-expanding venous reducer. To produce such a cylinder, braided material is initially shaped over a cylindrical mandrel. In an example, the Nitinol cylinder is heat set at temperatures of 400-550 degrees Celsius in fluidized baths of sand, ovens (static or conveyer) for a period of approximately 1 minute to 20 minutes depending on the heat capacity of the implant and the mold fixturing.

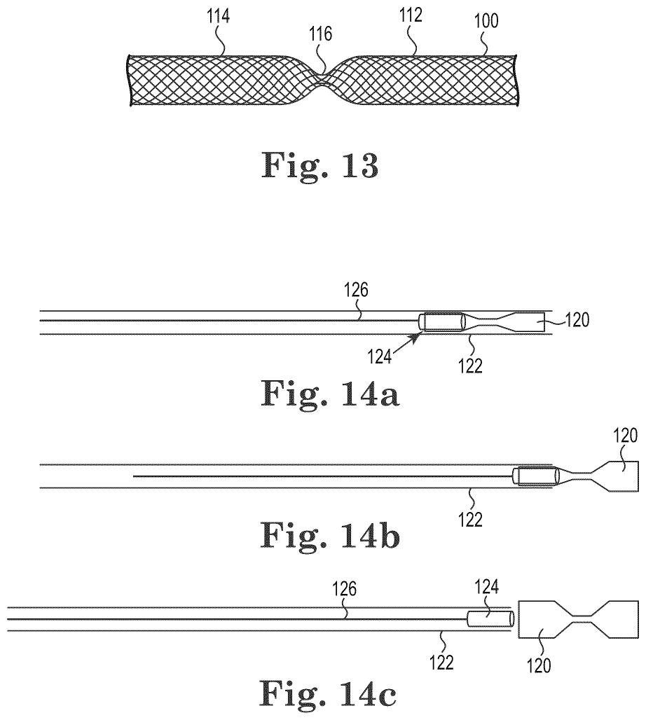

Braided cylinder 90 may then be placed onto a rod 101 having a relatively small diameter, as shown in FIG. 10 and bound in its central area with wire or heat-resistant thread 103. With such a method, forming tools 104 can be positioned within both ends of the cylinder 90 (i.e., on either side of the thread 103) to maintain the desired configuration of the cylindrical ends. In an alternative method, an hourglass-shaped mandrel 102 (see FIG. 12) may instead be positioned inside the cylinder 90, wherein the mandrel is provided in the general configuration of a final venous reducer 100, as is illustrated in FIG. 13.

The cylinder that has been shaped into a venous reducer can then be heat treated in a furnace or a fluidized bath. In an embodiment, the cylinder is heat treated in a thermolyne oven. In another embodiment, the fluidized bath includes alumina media and an air stream from the bottom that creates an easier surface into which the mold can be dipped.

The shaped venous stent 100 is then removed from the heat and quenched in room temperature water to set its shape. Proper quenching can better control the desired mechanical properties of the resultant stent, such as by controlling the austenitic finish temperature. The wire or heat-resistant thread can then be removed to provide a venous reducer 100 as shown in FIG. 13, which includes an upstream portion 112 and a downstream portion 114 extending at opposite ends from a narrow connection portion 116. This process would allow also for different shapes of the venous reducer, such as ends that flare at its distal ends, and also allow for different outer diameters that are customized to the patient and/or the area of the body in which the venous reducer will be implanted.

FIGS. 14a-14c are schematic views of exemplary steps for deploying a venous reducer using a fixed wire and resheathing pad with a delivery system. The delivery system would typically include a guidewire as is commonly used in many endovascular, venous, coronary, carotid, and neurovascular procedures. For venous applications, however, the venous anatomy is accessed percutaneously with an introducer and then a sequence of guidewires, sheaths, and guide catheters to access and cross the desired location. It is noted that the device can also be delivered retrograde. For CLI applications, for example, the device can be delivered from a pedal approach instead of via a femoral approach.

FIG. 14a illustrates an hourglass shaped venous reducer 120 positioned in a sheath or delivery catheter 122, a re-sheathing pad 124, and a pusher wire 126. The re-sheathing pad 124 can be a rubbery cylinder which engages with open areas of the venous reducer 120. FIG. 14b illustrates the pusher wire 126 as it has pushed the venous reducer 120 so that it extends partially from an end of the delivery catheter 122. In this configuration, it is still possible to adjust the position of the venous reducer. In FIG. 14c, the venous reducer 120 has been completely deployed beyond the end of the delivery catheter 122 and is now in an expanded condition.

A radiopaque marker (not shown) can be placed in the center of the venous reducer 129 to allow for more precise placement of the venous reducer at a valve location in the patient, for example. End markers may also be provided, as desired, such as to identify a landing zone that has less side branches. The venous reducer can be oversized to accommodate vessel tapering and/or may have a larger diameter on the stent end that will be positioned toward the vena cava. In general, the venous stents can be oversized to minimize migration as the vessels grow in diameter.

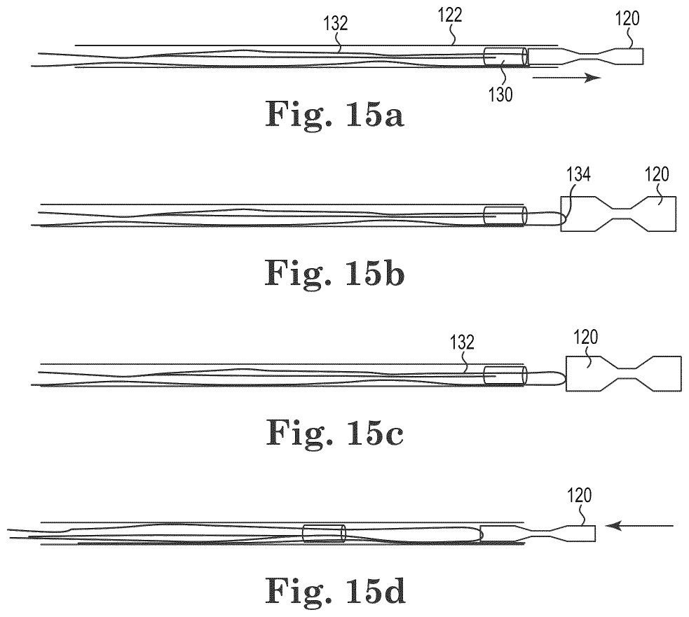

FIGS. 15a-15d are schematic views of exemplary steps for deploying a venous reducer using a stepped shoulder and tethers with a delivery system similar to that described relative to FIGS. 14a-14c. FIG. 15a illustrates an hourglass shaped venous reducer 120 positioned partially within a sheath or delivery catheter 122, a shoulder 130, and tethers 132 attached to the reducer 120. The shoulder 130 pushes the end of the venous reducer 120 out of the sheath and allows for adjustment prior to release. FIG. 15b illustrates the venous reducer 120 as it has been completely deployed beyond the end of the delivery catheter 122 and is now in an expanded condition. A suture loop 134 is visible within the venous reducer 120 in FIG. 15b. If adjustment of the venous reducer 120 is desired, the tether 132 can be pulled to tighten the suture loop, as is shown in FIG. 15c, and the venous reducer 120 can then be pulled back into the delivery catheter 122 as shown in FIG. 15d.

In general, the length of the delivery system and type of delivery system can be selected for different applications. For example, for shorter distance and single user systems, a rapid exchange system with a guidewire notch between the distal and proximal area can be useful. For longer travel system, typically for lower limb when accessing using a contralateral approach, an over-the-wire approach is used. For the coronary sinus, a reducer typically uses a jugular access which provides the benefit of a shorter distance; however, jugular access can more challenging for a number of reasons. Thus, femoral access is generally a preferred. A device designed for pedal access would need to have a reduced cross section (typically 4F or 5F sheaths). For ipsilateral or contralateral a size below 7F would allow small hole vessel closure. A pedal access may aid in the injection of x-ray contrast in fluoroscopy as the vein valves direct from back to the heart and this injection naturally flows in the direction.

The present invention has now been described with reference to several embodiments thereof. The entire disclosure of any patent or patent application identified herein is hereby incorporated by reference. The foregoing detailed description and examples have been given for clarity of understanding only. No unnecessary limitations are to be understood therefrom. It will be apparent to those skilled in the art that many changes can be made in the embodiments described without departing from the scope of the invention. Thus, the scope of the present invention should not be limited to the structures described herein, but only by the structures described by the language of the claims and the equivalents of those structures.

1. A method of treatment of critical limb ischemia within a foot of a patient, the method comprising the steps of:
delivering a venous reducer by way of the patient's vasculature to a first delivery location within a vein of the foot, the venous reducer comprising an expandable structure that when expanded includes an upstream portion and a downstream portion that are connected by a narrowed connection portion, and
expanding the venous reducer within the vasculature of the foot or ankle of the patient and thus creating an hourglass shaped structure within the vasculature where the narrowed connection portion acts as a restriction for blood flow.

2. The method of claim 1, wherein the step of expanding the venous reducer comprises expanding the venous reducer to a size and shape that improves arterial blood flow to the foot.

3. The method of claim 1, wherein the step of delivering a venous reducer comprises delivering the venous reducer to one of the foot or calf of the patient for regulation of a patient's venous plantar pump.

4. The method of claim 2, wherein expansion of the venous reducer causes higher pressure in a proximal portion of the venous plantar pump to: keep venous blood in the foot longer, thereby allowing remaining oxygen to be utilized and allowing oxygen to transfer across the capillaries at a higher pressure; drive pressure into the patient's microvasculature, thereby opening collapsed arteries and allowing better arterial blood flow into the foot; and increase oxygen in the foot.

5. The method of claim 1, further comprising the steps of:
maintaining the venous reducer in the vasculature of the foot or ankle of the patient for a desired treatment period after expanding the venous reducer; and
removing the venous reducer from the vein of the foot or ankle after the desired treatment period has elapsed.

6. The method of claim 1, wherein the narrowed connection portion increases fluid pressure within the patient's vasculature in the area of the upstream portion of the venous reducer.

7. The method of claim 1, wherein the delivery step comprises delivering the venous reducer to one of a plantar venous arch, a lateral plantar vein, a medial plantar vein, a posterior tibial vein, and an anterior tibial vein.

8. The method of claim 1, further comprising the steps of delivering at least one additional venous reducer to a second delivery location within a vein of the foot by way of the patient's vasculature.

9. The method of claim 1, wherein the step of expanding the venous reducer comprises one of:
simultaneously expanding both the upstream portion and the downstream portion; and
sequentially expanding the upstream portion and the downstream portion.

10. The method of claim 1, wherein the step of expanding the venous reducer comprises allowing self-expansion of the venous reducer from a compressed state.

11. The method of claim 1, wherein the expanded upstream portion comprises a diameter that is different from an expanded diameter of the downstream portion.

12. The method of claim 1, wherein the expanded upstream portion comprises a diameter that is the same as an expanded diameter of the downstream portion.

13. A method of treatment of critical hand ischemia within a hand or wrist of a patient, the method comprising the steps of:
delivering a venous reducer by way of the patient's vasculature to a delivery location within a vein of the hand or wrist, the venous reducer comprising an expandable structure that when expanded includes an upstream portion and a downstream portion that are connected by a narrowed connection portion, and
expanding the venous reducer within the vasculature of the hand or wrist of the patient and thus creating an hourglass shaped structure within the vasculature where the narrowed connection portion acts as a restriction for blood flow.

14. The method of claim 13, wherein the step of expanding the venous reducer comprises expanding the venous reducer to a size and shape that improves arterial blood flow to the hand.

15. The method of claim 13, wherein expansion of the venous reducer causes higher pressure in a portion of the hand to: keep venous blood in the hand longer, thereby allowing remaining oxygen to be utilized and allowing oxygen to transfer across the capillaries at a higher pressure; drive pressure into the patient's microvasculature, thereby opening collapsed arteries and allowing better arterial blood flow into the hand; and increase oxygen in the hand.

16. The method of claim 13, further comprising the steps of:
maintaining the venous reducer in the vasculature of the hand or wrist of the patient for a desired treatment period after expanding the venous reducer; and
removing the venous reducer from the vein of the hand or wrist after the desired treatment period has elapsed.

17. The method of claim 13, wherein the narrowed connection portion increases fluid pressure within the patient's vasculature in the area of the upstream portion of the venous reducer.

18. The method of claim 13, wherein the delivery step comprises delivering the venous reducer to one of a cephalic vein and a basilic vein.

19. The method of claim 13, further comprising the steps of delivering at least one additional venous reducer to a second delivery location within a vein of the hand by way of the patient's vasculature.

20. The method of claim 13, wherein the step of expanding the venous reducer comprises one of:
simultaneously expanding both the upstream portion and the downstream portion; and
sequentially expanding the upstream portion and the downstream portion.

21. The method of claim 13, wherein the step of expanding the venous reducer comprises allowing self-expansion of the venous reducer from a compressed state.

22. The method of claim 13, wherein the expanded upstream portion comprises a diameter that is different from an expanded diameter of the downstream portion.

23. The method of claim 13, wherein the expanded upstream portion comprises a diameter that is the same as an expanded diameter of the downstream portion.

24. A venous reducer for use in treatment of at least one of critical limb ischemia and critical hand ischemia, the venous reducer comprising:
an upstream portion sized and shaped for placement within the vasculature of at least one of a patient's hand or foot;
a downstream portion spaced from the upstream portion, wherein the downstream portion is sized and shaped for placement within the vasculature of a patient's hand or foot; and
a narrowed connection portion extending between the upstream portion and the downstream portion, wherein the narrowed connection portion comprises a diameter that is smaller than a diameter of the upstream and downstream portions.

25. The venous reducer of claim 24, wherein the upstream and downstream portions are expandable within the vasculature of at least one of a patient's hand or foot.

26. The venous reducer of claim 24, wherein the narrowed connection portion is not expandable.

27. The venous reducer of claim 24, comprising a bioabsorbable material that is absorbable into the vasculature of a patient at a desired time period after its placement within the vasculature of at least one of a patient's hand or foot.

28. A system for treating at least one of critical limb ischemia and critical hand ischemia, the system comprising:
a delivery system; and
a venous reducer comprising an expandable structure that when expanded includes an upstream portion sized and shaped for placement within the vasculature of a patient's hand or foot and a downstream portion sized and shaped for placement within the vasculature of a patient's hand or foot, and a narrowed connection portion connecting the upstream portion and the downstream portion.
